(19) United States
(12) Patent Application Publication
(10) Pub. No.: US 2011/0288101 A1
(43) Pub. Date: Nov. 24, 2011

(54) SURGICAL COMPOSITIONS CONTAINING SIGMA-RECEPTOR AGONISTS

(75) Inventors: Lixin Wang, Arlington, TX (US); Marcia Danielle Ong, Fort Worth, TX (US); Chi-Chun Tsai, Fort Worth, TX (US); Michael Hartzer, Arlington, TX (US)

(73) Assignee: ALCON RESEARCH, LTD., Fort Worth, TX (US)

(21) Appl. No.: 13/112,827

(22) Filed: May 20, 2011

Related U.S. Application Data
(60) Provisional application No. 61/347,173, filed on May 21, 2010.

(51) Int. Cl. A61K 31/439; C07C 211/28; A61K 31/137; C07C 251/58; A61P 41/00; C07D 307/14; A61K 31/341; C07D 295/096; A61K 31/495; A61P 27/02; C07D 221/26; A61K 31/15

(52) U.S. Cl. 514/252.12; 514/295; 546/97; 564/383; 514/654; 564/256; 514/640; 549/492; 514/471; 544/398

(57) ABSTRACT

One aspect of the present invention relates to aqueous surgical compositions comprising a sigma-1 receptor agonist. Another aspect of the present invention relates to methods of irrigating ocular tissues during an ophthalmic surgical procedure such as cataract surgery. Such methods comprise bathing the ocular tissues with a composition comprising a sigma-1 receptor agonist.

### CROSS-REFERENCE TO RELATED APPLICATION

This application claims priority under 35 U.S.C. §119 to U.S. Provisional Patent Application No. 61/347,173, filed May 21, 2010, the entire contents of which are incorporated herein by reference.

### TECHNICAL FIELD OF THE INVENTION

The present invention relates generally to surgical solutions and more specifically to ophthalmic surgical irrigating and viscoelastic solutions comprising a sigma-1 receptor agonist.

### BACKGROUND OF THE INVENTION

A wide variety of medical procedures, including wound cleansing, post-surgery adhesion prevention, debris removal from surgical fields, etc., rely on the use of surgical irrigating solutions. While many advanced surgical procedures minimize damage to tissues compared to older techniques, certain delicate procedures remain very sensitive to techniques and materials used. In particular, ophthalmic surgical procedures, such as cataract surgery and vitrectomy surgery, involve very fragile tissues (such as the corneal endothelium layer) and accordingly have little room for error and great potential for harm to such ocular tissues and the vision of the patient. Many of these procedures rely on the use of surgical irrigating solutions and viscoelastic solutions to protect delicate ocular tissues from trauma. Thus, there is an ongoing need to improve ophthalmic surgical techniques and equipment, as well as associated pharmaceutical products such as surgical irrigating and viscoelastic solutions.

Several literature references have reported that free radicals (such as peroxide radicals) can be produced in ocular tissue and structures such as the anterior chamber as a result of ocular surgery and associated procedures such as phacoemulsification (Takahashi et al., Arch Ophthalmology, Vol. 120:1348-1352, October 2002; Rubowitz et al., IOVS, Vol. 44(5):1866-1870, May 2003; Murano et al., Arch Ophthalmology, Vol. 126(6):816-821, June 2008; Shin et al., Arch Ophthalmology, Vol. 127(4):435-441, April 2009). These surgically-produced free radicals can damage delicate ocular tissues such as the corneal endothelium with resulting disruption of vision and ocular performance. Accordingly, methods to prevent oxidative damage caused by these free radicals are desirable.

The sigma-1 receptor, first cloned in 1996, is a single polypeptide transmembrane protein comprising 223 amino acids. It is mainly located on the endoplasmic reticulum membrane. Sigma-1 receptor is expressed in ocular tissues including the cornea, lens and retina, and has been reported to play a role in cell survival (Wang et al., Exp Cell Research, Vol. 312(8):1439-1446, 2006); Hayashi et al., Cell, Vol. 131(3):596-610, November 2007; Jiang et al., IOVS, Vol. 47(12):5576-5582, 2006).

Sigma receptor ligands have been found to be neuroprotective. The sigma receptor ligand opipramol was found to protect against ischemia in gerbils. In addition, other sigma ligands, including BMY-14802, caramiphen and haloperidol, exhibited properties in in vivo models that are consistent with protective effects (Pontecorvo et al., Brain Research Bulletin, Vol. 26:461-465, 1991). Several sigma ligands were found to inhibit ischemia-induced glutamate release from hippocampal slice preparations in vitro (Lobner et al., Neuroscience Letters, Vol. 117:169-174, 1990). It has also been shown that the Sigma-1 receptor agonist (+)-pentazocine can protect the retinal cells against stress (Dun et al., IOVS, Vol. 48(10):4785-4794, 2007; Smith et al., IOVS, Vol. 49(9):4154-4161, 2008). However, the literature does not describe the use of ophthalmically-compatible sigma-1 receptor agonists to protect against oxidative damage during ophthalmic surgical procedures.

### BRIEF SUMMARY OF THE INVENTION

Embodiments of the present invention are directed to surgical compositions comprising a sigma-1 receptor agonist. In a preferred embodiment, the present invention relates to ophthalmic irrigating or viscoelastic compositions comprising the sigma-1 receptor agonist (+)-pentazocine.

Yet another embodiment of the present invention relates to methods of irrigating ocular tissues during a surgical procedure, which comprises bathing the intraocular tissues with a surgical composition comprising a sigma-1 receptor agonist. In a preferred embodiment, the sigma-1 receptor agonist is (+)-pentazocine.

The sigma-1 receptor agonist (+)-pentazocine has minimal toxic effect on ocular tissues. It protects the corneal endothelial cells by activating Sigma-1 receptor and does not appear to cause any deleterious cellular proliferation and/or morphological changes

The compositions of the present invention are suitable for use in a variety of ophthalmic and non-ophthalmic surgical procedures, but are particularly adapted and well-suited for use in conjunction with ophthalmic surgical procedures. The compositions are especially useful in conjunction with anterior chamber ophthalmic procedures that have the potential to expose the endothelial cells of the cornea. In other applications, the compositions may be used for foreign body removal and washing procedures. Certain compositions of the present invention are suitable for posterior chamber procedures such as vitrectomy and for procedures involving the retina. The above list is not comprehensive and those of skill in the art will appreciate other applications for the disclosed embodiments of the present invention.

Yet another embodiment of the present invention is a method of protecting endothelial cells during a surgical procedure comprising administering a composition having a sigma-1 agonist. The composition is administered in a manner to ensure contact between the composition and the endothelial cells to be protected. In a preferred embodiment, the composition is administered to make contact with vascular endothelial cells using intravenous administration.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with specific embodiments described herein. However, the specific examples provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings and wherein:

FIG. 1 is graph showing the effects of (+)-pentazocine on cell viability in an in vitro experiment using rabbit corneal endothelial cells.

### DETAILED DESCRIPTION OF THE INVENTION

The compositions of the present invention are generally aqueous, isotonic electrolyte or viscoelastic solutions that have a physiologically-compatible pH. The compositions comprise a sigma-1 receptor agonist such as (+)-pentazocine. In other embodiments, sigma-1 receptor agonists such as AGY-94806, AE-37 (tetrahydro-N,N-dimethyl-2,2-diphenyl-3-furanomethanamine), igmesine, fluvoxamine, and others known to those of skill in the art may be used.

Methods for identifying additional compounds that are sigma receptor ligands are known in the art. One method used to identify compounds that are ligands for the sigma receptor involves placing cells, tissues, or preferably a cellular extract or other preparation containing sigma receptors in contact with several known concentrations of a test compound in a buffer compatible with receptor activity, and assaying for ligand binding and/or receptor activity. The method can be performed either sequentially or in a multiplexed format. The use of in vitro binding assays with known specific ligands can allow for the determination of ligand affinities for sigma-1 or sigma-2 receptors as described in Langa, et al., European J. Neuroscience, Vol. 18:2188-2196, 2003.

The preferred sigma-1 receptor agonists of the present invention have little or no toxicity when applied topically to ocular tissues. Sigma-1 receptor agonists can be screened for toxicity using in vitro and in vivo assays known to those of skill in the art.

The concentration of the sigma-1 receptor agonists in the compositions of the present invention may vary, but is generally between 0.01 w/v % to 1.0 w/v %, preferably 0.01 to 0.05 w/v % and most preferably about 0.01 to 0.03 w/v %.

The solutions of the present invention may further comprise a variety of excipients such as buffering systems, essential ions, polymers, viscosity adjusting agents, energy sources, etc. A variety of buffering systems known to those of skill in the art may be used with embodiments of the invention. In some embodiments of the present invention, bicarbonate or phosphate buffering systems provide adequate buffering capacity to maintain pH. Citrate buffering systems may also be used with certain embodiments of the present invention.

Viscosity adjusting agents such as chondroitin sulfate, sodium hyaluronate or other proteoglycans; cellulose derivatives, such as hydroxypropyl methylcellulose (“HPMC”), carboxy methylcellulose (“CMC”), and hydroxyethyl cellulose (“HEC”); collagen and modified collagens; galactomannans, such as guar gum, locust bean gum and tara gum, as well as polysaccharides derived from the foregoing natural gums and similar natural or synthetic gums containing mannose and/or galactose moieties as the main structural components (e.g., hydroxypropyl guar); xanthan gum; gellan gums; alginate; chitosans; polyvinyl alcohol; carboxyvinyl polymers (e.g., carbomers such as CARBOPOL®); and various other viscous or viscoelastomeric substances may be used with embodiments of the present invention.

The solutions of the present invention may further comprise carbohydrate energy sources, such as polysaccharides (e.g., sucrose) or monosaccharides (e.g., dextrose).

Compositions of the present invention may comprise essential ions such as sodium, potassium, and chloride. Potassium and sodium may be provided in the form of various sodium and potassium salts known to those of skill in the art, such as sodium or potassium chlorides, sulfates, acetates, citrates, lactates, and gluconates. Similarly, chloride salts, such as sodium chloride and magnesium chloride, may be used to provide chloride in solutions of the present invention. For the essential ions, the concentration of potassium should be about 0.01 w/v % to about 0.5 w/v %, with the most preferred concentration about 0.04 w/v %. The concentration of sodium should be about 0.1 w/v % to about 1.0 w/v %, with the most preferred concentration about 0.55 w/v %.

Ophthalmic irrigating compositions of the present invention may comprise commercially available solutions such as BSS® (Alcon, Inc) combined with a sigma-1 receptor agonist such as (+)-pentazocine. Other irrigating solutions are known to those of skill and described in publications such as U.S. Pat. No. 7,084,130 to Shah et al. (herein incorporated by reference in its entirety).

In other embodiments of the present invention a sigma-1 receptor agonist is combined with excipients to form viscoelastic compositions demonstrating desirable rheological behavior (e.g., pseudo-plastic behavior, non-Newtonian flow, etc.). Examples of such viscoelastic compositions are commercially available (e.g., VISCOAT®, DISCOVISC®, and PROVISC®, Alcon, Inc.) and described in various publications (e.g., U.S. Pat. No. 6,051,560 to Chang et al., herein incorporated by reference in its entirety).

In preferred embodiments of the present invention, the compositions are prepared by mixing all ingredients and stirring until all components have entered solution. The solution is then sterilized by dry or steam heat for a set time period (typically 30 minutes at 121° C.). However, the time and temperature of sterilization may vary and can be optimized by those of skill in the art. Other sterilization techniques known to those of skill may be necessary if one or more components of the composition are heat labile.

In embodiments for the treatment of systemic endothelial tissues such as vascular endothelial cells, various administration techniques known to those of skill in the art may be used. For example, a sigma-1 agonist can be formulated for intravenous injection in a sterile electrolyte or glucose solution.

The compositions described in Examples 1 and 2 below were prepared according to embodiments of the present invention and are provided to further illustrate various features of the present invention.

### Example 1

| Ingredient | (% w/v) |
|---|---|
| Sigma-1 Receptor Antagonist | 0.4 |
| Sodium Chloride | 0.744 |
| Potassium Chloride | 0.0395 |
| Dibasic Sodium Phosphate, Anhydrous | 0.0433 |
| Sodium Bicarbonate | 0.22 + 10% excess |
| Hydrochloric Acid and/or Sodium Hydroxide | adjust to pH 6.8 |
| Water for Injection | q.s. to 100 |

### Example 2

| Ingredient | (w/v %) |
|---|---|
| (+) pentazocine (10 μM) | 0.029 |
| Sodium Hyaluronate | 3.0 |
| Sodium Chondroitin Sulfate | 4.0 |
| Monobasic Sodium Phosphate, Monohydrate | 0.045 |
| Dibasic Sodium Phosphate, Anhydrous | 0.20 |
| Sodium Chloride | 0.43 |
| Sodium Hydroxide and/or Hydrochloric Acid | pH Adjust to 7.2 |
| Water for Injection | q.s. to 100 mL |

### Example 3

Studies were performed to determine whether (+)-pentazocine can protect corneal endothelial cells. Hydrogen peroxide was employed to produce oxidative stress to rabbit corneal endothelial cells in vitro.

Briefly, rabbit corneal endothelial cells maintained in serum-free medium were treated with the sigma-1 receptor agonist (+)-pentazocine and 70 μM H2O2 for 24 hours. At the end point, a cell viability assay (MTS assay) is performed. The MTS assay is a laboratory test and standard colorimetric assay for measuring the activity of enzymes that reduce MTS+PMS to formazan, producing a purple color. MTS (3-(4,5-dimethylthiazol-2-yl)-5-(3-carboxymethoxyphenyl)-2-(4-sulfophenyl)-2H-tetrazolium), in the presence of phenazine methosulfate (PMS), produces a water-soluble formazan product that has an absorbance maximum at 490-500 nm in phosphate-buffered saline. These reduction reactions take place only when reductase enzymes are active, and the production of formazan product is therefore used as a measure of cell viability.

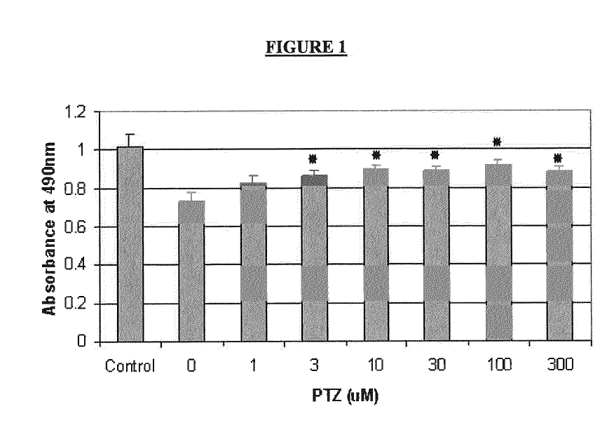

The results of the experiment are presented in FIG. 1. Lower absorbance numbers correlate to reduced cell viability. The bar graph shows that (+)-pentazocine (abbreviated PTZ on the x-axis) provides dose-dependent protection for the rabbit corneal endothelial cells against damage induced by hydrogen peroxide (70 μM). The results are statistically significant, and represent a demonstrated difference between the 70 μM hydrogen peroxide treated control group without (+)-pentazocine (0) and the experimental groups (1 to 300 μM (+)-pentazocine) (t-test value p<0.05).

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. An ophthalmic composition comprising a sigma-1 receptor agonist.

2. A composition of claim 1 wherein said sigma-1 receptor agonist is selected from the group consisting of:
(+)-pentazocine, AGY-94806, AE-37 (tetrahydro-N,N-dimethyl-2,2-diphenyl-3-furanomethanamine), igmesine, and fluvoxamine.

3. A composition of claim 1 wherein the sigma-1 receptor agonist is (+)-pentazocine.

4. A composition of claim 1 wherein the concentration of the sigma-1 agonist is 0.01 w/v % to 1.0 w/v %.

5. A composition of claim 1 wherein the concentration of the sigma-1 agonist is 0.01 w/v % to 0.03 w/v %.

6. A method of irrigating ocular tissues during a surgical procedure comprising:
bathing the ocular tissues with an irrigating composition of claim 1.

7. A method according to claim 6 wherein said composition comprises sodium hyaluronate and chondroitin sulfate.

8. A method according to claim 6 wherein the sigma-1 receptor agonist is (+) pentazocine.

9. A method according to claim 6 wherein the concentration of the sigma-1 agonist is 0.01 w/v % to 1.0 w/v %.

10. A composition of claim 6 wherein the concentration of the sigma-1 agonist is 0.01 w/v % to 0.03 w/v %.

11. A method of protecting endothelial cells during a surgical procedure comprising:
administering a composition of claim 1, wherein said administering causes said composition to contact the endothelial cells to be protected.

12. A method according to claim 11 wherein said endothelial cells are vascular or corneal endothelial cells.

13. A method according to claim 11 wherein the sigma-1 receptor agonist is (+) pentazocine.

14. A method according to claim 11 wherein the concentration of the sigma-1 agonist is 0.01 w/v % to 1.0 w/v %.

15. A composition of claim 11 wherein the concentration of the sigma-1 agonist is 0.01 w/v % to 0.03 w/v %.
